(19) United States
(12) Patent Application Publication
(10) Pub. No.: US 2020/0038355 A1
(43) Pub. Date: Feb. 6, 2020

(54) ANTIOXIDANTS FOR USE IN OPHTHALMIC SURGERY

(71) Applicant: Case Western Reserve University, Cleveland, OH (US)

(72) Inventors: Xingjun Fan, Cleveland, OH (US); Zongbo Wei, Cleveland, OH (US)

(21) Appl. No.: 16/605,814

(22) PCT Filed: Apr. 30, 2018
(86) PCT No.: PCT/US18/30118

§ 371 Date: Oct. 17, 2019

Related U.S. Application Data
(60) Provisional application No. 62/491,574, filed on Apr. 28, 2017.

(51) Int. Cl. A61K 31/198; A61K 31/7084

(52) U.S. Cl. CPC A61K 31/198; A61K 9/0051; A61K 31/7084

(57) ABSTRACT

A method of decreasing eye oxidation is described. The method includes administering a therapeutically effective amount of an antioxidant to a subject undergoing an ophthalmic surgical procedure.

### CONTINUING APPLICATION DATA

This application claims the benefit of U.S. Provisional Application Ser. No. 62/491,574, filed Apr. 28, 2017, all of which is incorporated by reference herein.

### GOVERNMENT FUNDING

This invention was made with government support under Grant No. EY024553, awarded by the National Institutes of Health. The government has certain rights in this invention.

### BACKGROUND

Cataract surgery remains the only effective procedure to treat cataracts at an estimated worldwide rate of 30 million procedures each year. Even though cataract surgery is a highly safe and effective medical procedure, it often carries complications. Posterior capsule opacification (PCO) is the most common complication postoperatively. Proliferation, migration, and transdifferentiation of residual lens epithelial cells following surgery, subsequently causing posterior capsular opacification (PCO) and wrinkling that affects visual acuity, a condition also known as secondary cataract.

PCO formation is thought to originate via a fibrotic process involving wound healing and tissue-remodeling pathways initiated by traumatic injury during the surgical procedure. Growing evidence indicates that epithelial-mesenchymal transition (EMT) of lens epithelial cells plays a key pathogenic role in PCO formation (Mamuya et al., J Cell Mol Med 18, 656-670 (2014)), which has also been very well documented in organ fibrotic diseases, such as kidney (He et al., Clin Exp Nephrol 17, 488-497 (2013)), lung, and liver fibrosis (Lee et al., World J Hepatol 6, 207-216 (2014)). During EMT, lens epithelial cells lose their tight junction molecules and transdifferentiate into mesenchymal cells, a cell type with myofibroblast morphology that is more invasive and no longer maintains monolayer characteristics. Furthermore, pro-mesenchymal cytoskeletal protein overproduction, including alpha smooth muscle actin (αSMA) and other extracellular proteins, also contribute to posterior capsule wrinkling. Marcantonio et al., Exp Eye Res 77, 339-346 (2003). EMT is thought to be triggered by inflammatory cytokines and basement membrane proteolysis via metalloproteinases. Lamouille et al., Nat Rev Mol Cell Biol 15, 178-196 (2014) The elevated expression of TGFβ, fibroblast growth factor (FGF), hepatocyte growth factor (HGF), epidermal growth factor (EGF), insulin-like growth factor (IGF) can engage the EMT process via receptor and intrinsic kinase-mediated signal transduction. Extensive studies from cell and ex vivo capsular bag culture models suggest that TGF3 is a critical regulator of lens EMT pathogenesis, which is mediated by a Smad dependent (Saika et al., Br J Ophthalmol 86, 1428-1433 (2002)) and a Smad independent (Tiwari et al., Invest Ophthalmol Vis Sci 56, 272-283 (2014)) signaling cascade. This results in up-regulation of various profibrotic genes, including fibronectin, type I&III collagen, α-smooth muscle actin (aSMA), vimentin and desmin, matrix metalloproteinase 2 and 9 (MMP-2, MMP-9), and integrins, such as α5, all and αVβ5.

Moderate chronic oxidative stress has been suggested to play a pivotal role in organ fibrosis as well as tumor metastasis. Morry et al., Redox Biol 11, 240-253 (2017). Reactive oxygen species (ROS) and oxidative stress have also been implicated in modulating EMT signaling. Cannito et al., Antioxid Redox Signal 12, 1383-1430 (2010). Mori K. et al. found that the mouse mammary gland epithelial cell line NMuMG could convert to a fibroblast-like cell type after several days of challenge by a low dosage of H₂O₂. Mori et al., Cancer Res 64, 7464-7472 (2004). This transformation was accompanied by an upregulation of integrins (α2, α6, and β3) and several MMPs, and an E-cadherin redistribution via a MAPK signaling cascade. A similar study was reported in HK-2 normal kidney epithelial cell malignant transformation under a noncytotoxic dose of H₂O₂ that resulted in significant up-regulation of mesenchymal markers, such as β-catenin, vimentin, and αSMA. Mahalingaiah et al., J Cell Physiol 230, 1916-1928 (2015). The reciprocal interplay between ROS and the most potent profibrogenic cytokine, TGFβ, critically modulate cell proliferation, extracellular matrix deposition, and cell transdifferentiation. Droge, W., Physiol Rev 82, 47-95 (2002). This is well supported by observations of NADPH oxidase, a ROS production enzyme, upregulation in various fibrotic disease. Amara et al., Thorax 65, 733-738 (2010). Furthermore, significant declines in GSH levels have been documented in experimental fibrosis models (Iyer et al., Am J Physiol Lung Cell Mol Physiol 296, L37-45 (2009)) as well as in various human fibrotic diseases, including cystic fibrosis (Roum et al., J Appl Physiol (1985) 75, 2419-2424 (1993)), pulmonary fibrotic disease (Rahman et al., Free Radic Biol Med 27, 60-68 (1999)), and liver fibrosis (Bianchi et al., J Hepatol 26, 606-613 (1997)). However, the pathogenic role of ROS and oxidative stress in lens epithelial cell fibrosis, as well as in PCO formation after cataract surgery, remains unknown.

TGFβ is recognized as a key mediator in lens epithelial cells fibrosis in PCO formation. De Longh et al., Cells Tissues Organs, 179, 43-55 (2005) However, despite strong attenuation of the EMT signaling by blocking either TGFβ or its downstream pathways at in vitro cell culture or ex vivo human capsular bag culture models, no effect was found in an in vivo rodent PCO model when treated with anti-TGFβ2 antibody. Lois et al., Invest Ophthalmol Vis Sci 46, 4260-6 (2005) Over the years, clinical trials of anti-inflammatory and apoptosis-inducing drugs did not result in promising outcomes for preventing posterior capsule opacification resulting from cataract surgery. Accordingly, there is an unmet need for a satisfying pharmacological intervention to prevent eye oxidation following ophthalmic surgery.

### SUMMARY OF THE INVENTION

The present invention provides methods of decreasing eye oxidation by administering a therapeutically effective amount of an antioxidant to a subject undergoing an ophthalmic surgical procedure.

Chronic oxidation promotes EMT signaling and extracellular matrix deposition based on in vitro cell culture, ex vivo mouse lens culture, ex vivo lens explant culture, and in vivo mouse mock cataract surgery experimental models. The reduced GSH biosynthesis or GSH concentration-based mouse (Gclc and Gclm knockout) and cell culture experimental models study indicates a significant increased production of pro-EMT markers, such as type I collagen, alpha smooth muscle actin (αSMA), vimentin, and fibronectin. Further study suggests the oxidative stress from decreased GSH triggers the Wnt/β-catenin signal transduction pathway and affects the β-catenin destruction complex via phosphorylation/inactivation of serine 9 of GSK-3β. The oxidative stress modulated epithelial-mesenchymal transition (EMT) signaling is not because of TGFβ2 and its canonical pathway. These results were further confirmed in the in vivo mouse mock cataract model, and a PCO mouse model.

Thiol-based antioxidants could significantly attenuate both oxidative stress and TGF-β2-induced EMT signaling and extracellular matrix protein production. Equally important, the antioxidant N-acetyl cysteine (NAC) and glutathione ethyl ester (GSH-EE) could significantly attenuate the EMT signaling stimulated by either TGFβ2 or decreased GSH levels. These findings are further confirmed in mouse mock cataract surgery in vivo in both Gclc and Gclm knockout experimental models. The remarkably increased EMT marker expression is found in both Gclc and Gclm knockout mice compared to wild type and such increased expression could be significantly attenuated by NSC or GSH-treatment.

### BRIEF DESCRIPTION OF THE FIGURES

FIGS. 1A and 1B provide images showing the profound environmental change that occurs in the lens epithelial cells before and after cataract surgery. A) Lens epithelial cells apical side is in contact with a GSH-enriched and closed microenvironment system. B) Lens epithelial cells apical side faces GSH-depletion and an open microenvironment.

FIGS. 2A-2F provide graphs and images showing EMT markers are enhanced in LEGSKO and GCLM KO mice at 4 months. Lens or cell homogenate were analyzed by immunoblot probing αSMA, vimentin, GCLC, GCLM, and GAPDH. (A) αSMA is in the whole lens homogenate of LEGSKO mice compared to WT (p<0.05). (B) αSMA is increased in LEGSKO lens capsule compared to WT (p<0.01). (C) αSMA is increased in LEGSKO lens fiber compared to WT (p<0.05). (D) αSMA and vimentin are increased in the whole lens homogenate of Gclm KO mice compared to WT (p<0.05). (E) αSMA and vimentin levels are increased in Gclm KO mice lens capsule compared to WT (αSMA, p<0.05; vimentin, p<0.01). (F) αSMA is are increased in Gclm KO mice lens fiber compared to WT (p<0.05). All results are the mean±SEM. Whole lens, n=6; capsule or fiber, n=3. GAPDH was used as a loading control. GCLC and GCLM blotting served as additional genotype verification beside PCR genotype conclusion. Student t test was used to compare WT and KO group, and p<0.05 is considered as significant.

FIGS. 3A-3H provide graphs and images showing EMT markers are elevated under GSH depletion condition. (A) αSMA expression is increased in HLE-B3 cells after treatment with increased concentration of BSO for 24 hr. (B) αSMA expression is increased in HLE-B3 cells after treatment with increasing concentration of dimethylformamide (DMF) for 24 hr. (C) Significantly increased expression of collagen I, vimentin and αSMA is seen in 500 μM BSO, 25 μM DMF and 500 μM BSO+25 μM DMF stimulated cells for 24 hr, respectively (p<0.01, or p<0.001 as indicated inside graph). (D) The intracellular GSH concentration after 24 hr treatment with 500 μM BSO, 25 μM DMF or 500 μM BSO combined with 25 μM DMF. (E) αSMA expression increases in pLECs after treatment with increased concentration of BSO for 24 hr. (F) Increased αSMA and vimentin expression is seen in the lens capsule from cultured whole lens treated with 500 μM BSO for 48 hr compared to medium alone (p<0.01). (G) αSMA production is alleviated in HLE-B3 cells when co-treated with either 10 mM NAC, 0.5M GSH-EE with BSO (500 rμM) for 24 hr, respectively (p<0.05, p<0.001, see label in graph). (H) αSMA production is alleviated in the lens capsule from cultured whole lens when co-treated with 10 mM NAC, 0.5 mM GSH-EE with 500 μM BSO for 48 hr, respectively (p<0.01, p<0.001, see label in graph). GAPDH was used as a loading control and n=5 for each immunoblot calculation. One-way ANOVA and Student t test was used to compare WT and KO groups, and p<0.05 is considered as significant.

FIGS. 4A-4H provide graphs and images showing TGF-β-smad signaling is not altered under GSH depletion condition. (A) No remarkable changes of p-smad2 and p-smad3 is seen in HLE-B3 cells challenged by 500 μM BSO for 24 hr or lens capsule from Gclm KO mice compared to WT. (B) Synergistic effect is seen in HLE-B3 cells challenged by 500 μM BSO, 1 ng/ml TGFβ2, 500 μM BSO+1 ng/ml TGFβ2 for 24 hr, respectively. (C) αSMA production is alleviated by 10 mM NAC (p<0.001) or 0.5 mM GSHEE (p<0.05) in lens capsule from whole lens culture when co-incubated with 1 ng/ml TGFβ2 for 48 hr. (D) αSMA production is alleviated by 10 mM NAC (p<0.001) or 0.5 mM GSH-EE (p<0.05) in HLE-B3 cells when co-incubated with 1 ng/ml TGFβ2 for 24 hr. (E-H) αSMA expression is activated in lens explant culture after 48 hr stimulation by TGFβ2 compared to medium alone, and such activation could be attenuated by co-incubation with either 10 mM NAC or 0.5 mM GSH-EE. For immunoblot, all data calculated have been adjusted by GAPDH level and n=5 in each assay. One-way ANOVA and Student t test was used to compare WT and KO groups, and p<0.05 is considered as significant.

FIGS. 5A-5M provide graphs and images showing oxidative stress modulated EMT signaling is mediated via the Wnt/β-catenin signaling pathway. (A) Wnt 10a and β-catenin are (p<0.01) elevated in the whole lens protein extract in both LEGSKO and Gclm KO mice compared to WT. (B) Similarly, Wnt 10a and β-catenin are significantly (p<0.01) elevated in the lens capsule protein extract in both LEGSKO and Gclm KO mice compared to WT. (C) The αSMA and β-catenin production is blocked when XAV939, a Wnt inhibitor is co-incubated with 500 μM BSO in lens ex vivo culture (lens capsule was extracted for immunoblot analysis) and HLE-B3 cells. (D) The serine 9 phosphorylation of GSK-3β (p-GSK-3βSer9) is significantly elevated in LEGSKO lens capsule compared to WT (p<0.01) and also in HLE-B3 cells after stimulation with 500 μM BSO for 24 hr (p<0.05). GSH-EE and NAC attenuate β-catenin expression and GSK-3βSer9phosphorylation, respectively. (E-M) Immunofluorescence image of activate β-catenin colocalization within the nucleus. The active β-catenin, Tyr489 phosphorylated β-catenin was labeled in green, nucleus was labeled in blue by DPAI. The inset image is enlarged for display. The white dotted line illustrated the boundary of lens capsule. (E-G) WT lens capsule 72 hr after surgery demonstrates mild β-catenin nuclear translocation. (H-J) LEGSKO mice lens capsule demonstrates robust nucleus translocation of β-catenin. (K-M) The active β-catenin nuclear translocation was significantly attenuated when 10 mM NAC is applied immediately after surgery. For immunoblot, all data have been adjusted for GAPDH level and n=5 in each assay. One-way ANOVA and Student t test was used to compare WT and KO groups, and p<0.05 is considered as significant.

### DETAILED DESCRIPTION OF THE INVENTION

### Definitions

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein and in the appended claims, the singular forms “a”, “and”, and “the” include plural referents unless the context clearly dictates otherwise. Thus, for example, reference to “a sample” also includes a plurality of such samples and reference to “the splicing regulator protein” includes reference to one or more protein molecules, and so forth.

“Biocompatible,” as used herein, refers to any material that does not cause injury or death to the animal or induce an adverse reaction in an animal when placed in intimate contact with the animal's tissues. Adverse reactions include for example inflammation, infection, fibrotic tissue formation, cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, does not cause prolonged inflammation or irritation, or does not induce more than a basal immune reaction in the host.

As used herein the term “polynucleotide” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Natural nucleic acids have a deoxyribose- or ribose-phosphate backbone.

As used herein, the term “subject” is inclusive of both human and animal subjects. Thus, the present invention provides methods for preventing oxidative damage in mammals such as humans, as well as other mammals. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like. Where the subject is human, in some embodiments the method is used to decrease eye oxidation in subjects of a specific age group, such as infant or elderly subjects. Examples of elderly subjects, who have an increased risk of developing eye oxidation, include subjects that are 50 years or older, as 60 years or older or 70 years or older.

### Methods of Decreasing Eye Oxidation

In one aspect, the present invention provides a method of decreasing eye oxidation. The method includes administering a therapeutically effective amount of an antioxidant to a subject undergoing an ophthalmic surgical procedure. “Undergoing” an ophthalmic surgical procedure, as used herein, includes subjects who will soon undergo an ophthalmic surgical procedure, subjects who are currently undergoing an ophthalmic surgical procedure, and subjects who have recently undergone an ophthalmic surgical procedure. In some embodiments, the method of decreasing oxidative damage comprises administering the antioxidant to the eye of a subject. In some embodiments, the composition is administered to a subject after an ophthalmic surgical procedure to decrease the amount of posterior capsule opacification that may otherwise be experienced by the subject and that may potentially lead to the development of secondary cataracts.

The terms “decrease,” “decreasing,” or “decreased” when used herein refer to any reduction or suppression in the amount or rate of oxidative damage to the eye of a subject, such as the eye lens. Of course, it is understood that the amount of the decrease need not be absolute (i.e., the degree of inhibition need not be a complete prevention of oxidative damage) and that intermediate levels of a reduction in oxidative damage are contemplated by the present invention. As such, in some embodiments, the decrease in oxidative damage can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In some embodiments, the method includes reducing the occurrence of opacity or cataracts (e.g., secondary cataracts) in the eye, e.g., as assessed relative to any convenient control. In certain embodiments, the occurrence of a cataract is assessed by slit-lamp examination, or by a visual acuity measurement. In some instances, reduction in the occurrence of opacity or cataracts in the eye that is achieved in the subject methods is assessed by measuring a period of time post-surgery in which no opacity is observed in the eye. In some cases, the subject methods prevent or delay the onset of opacity or cataracts in the eye for at least 1 week, such as at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 1 year, at least 2 years, at least 5 years, at least 10 years, or even indefinitely.

The present invention provides a method of decreasing eye oxidation. Oxidation can be decreased in one or more components of the eye. The major structures of the eye are the sclera, the cornea, the iris, the lens, the vitreous, the retina, and the posterior capsule. The eye also includes epithelial tissue in various regions. The present invention can be used to decrease eye oxidation in any region of the eye. In some embodiments, the method is used to decrease oxidation of the posterior capsule in order to decrease posterior capsule opacification.

### Antioxidants

The method includes administering a therapeutically effective amount of an antioxidant to a subject. “Antioxidants,” as used herein, refer to substances capable of inhibiting oxidation of molecules or, in other words, substances capable of inhibiting the transfer of electrons or hydrogen from a particular substance to an oxidizing agent. In some embodiments, the term “antioxidant” can thus be used interchangeably with the term “oxygen quenching substance.” Preferably, the antioxidant is a “strong” antioxidant, that is, one with demonstrated high potency. Antioxidants include small molecule antioxidants, as well as antioxidant enzymes or antioxidant enzyme activators, such as polynucleotides that are effective to increase antioxidant enzyme activity through gene therapy.

Small molecule antioxidants are compounds that can be readily obtained or prepared through organic synthesis methods, although in some cases they can be obtained from natural sources. Because they are being used therapeutically, the antioxidants should be biocompatible. Small molecule antioxidants include organic compounds and minerals having antioxidant activity. Small molecule antioxidants preferably have a molecular weight ranging from about 50 to about 5000 daltons, with some embodiments being directed to antioxidants having a weight ranging from about 100 to about 4000 daltons, or from about 200 to about 3000 daltons. Examples of small molecule antioxidants include glutathione, glutathione-ethyl ester, N-acetyl cysteine, cysteine, curcumin, zinc, selenium, tocopherols, beta-carotene, lutein, zeaxanthin, lycopene, allyl sulfide, and various indoles. Other antioxidants which are known or are developed can also be administered. While a single antioxidant is typically administered, in some embodiments, a plurality of different antioxidants can be administered to the subject.

In some embodiments, the small molecule antioxidants are thiol-based antioxidants, which are small molecules including a thiol group. For a comprehensive list of thiol-based antioxidants, see Deneke S. M., Current Topics in Cellular Regulation, 36, 151-180 (2001), the disclosure of which is incorporated herein by reference. Preferred thiol-based antioxidants for use in decreasing eye oxidation include N-acetyl cysteine and glutathione ethyl ester.

While the method of decreasing eye oxidation can involve the use of other active agents, in some embodiments, treatment consists of administration of the antioxidant without other active agents being present, such that treatment consists essentially of administration of the antioxidant. Exclusion of other active agents does not exclude the presence of a pharmaceutically acceptable carrier, and therefore in some embodiments treatment consists or consists essentially of administration of the antioxidant together with a pharmaceutically acceptable carrier.

### Antioxidant Enzyme Activators

In some embodiments, the antioxidant is an antioxidant enzyme activator. Antioxidant enzyme activators are agents that increase the activity of an antioxidant enzyme. A variety of antioxidant enzymes are known to those skilled in the art. Antioxidant enzymes can also be selected that are typically found in the eye. In some embodiments, the antioxidant enzyme is superoxide dismutase (DOS), catalase, glutathione peroxidase, or glutathione reductase.

In some embodiments, the antioxidant enzyme activators are polynucleotides that are administered by gene therapy. In such embodiments, the antioxidant enzyme activator is a polynucleotide that can stimulate increased expression of an antioxidant enzyme. Gene therapy can use an expression vector including a polynucleotide encoding an antioxidant enzyme. An “expression vector” (sometimes referred to as gene delivery or gene transfer “vehicle”) refers to a macromolecule or complex of molecules comprising a polynucleotide to be delivered to a target cell to cause in vivo expression of the antioxidant enzyme. Preferred target cells for the present invention include cells within the eye. Vectors include, for example, viral vectors (such as adenoviruses (‘Ad’), adeno-associated viruses (AAV), and retroviruses), liposomes and other lipid-containing complexes, and other macromolecular complexes capable of mediating delivery of a polynucleotide to a target cell. Use of vectors to administer a polynucleotide capable of expressing superoxide dismutase, catalase, or glutathione peroxidase have been described. See US Patent Publication No. 2011/0082191, US Patent Publication No. 2011/0136197, and U.S. Pat. No. 7,241,591, the disclosures of which are incorporated herein by reference.

Expression vectors for use in the present invention include viral vectors, lipid based vectors and other non-viral vectors that are capable of delivering a nucleotide according to the present invention to the target cells. The expression vector can be a targeted vector, especially a targeted vector that preferentially binds to cells of proximate the wound. Viral vectors for use in the invention can include those that exhibit low toxicity to a target cell and induce production of therapeutically useful quantities of an antioxidant enzyme in a tissue-specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the antioxidant enzyme (e.g., superoxide dismutase) and is replication-defective in humans. Methods of using adeno-associated virus for gene therapy of the eye have been described. See US Patent Publication No. 2016/0361439, the disclosure of which is incorporated herein by reference.

Other viral vectors that can be use in accordance with the present invention include herpes simplex virus (HSV)-based vectors. HSV vectors in which one or more immediate early genes (IE) have been deleted are advantageous because they are generally non-cytotoxic, persist in a state similar to latency in the target cell, and afford efficient target cell transduction. Recombinant HSV vectors can incorporate approximately 30 kb of heterologous nucleic acid.

One or more promoters can be included in the expression vector to increase expression of the heterologous gene to be expressed by the expression vector. Further, the expression vector can comprise a sequence which encodes a signal peptide or other moiety which facilitates the secretion of an antioxidant enzyme from the target cell. Other nucleotide sequence elements which facilitate expression of a gene expressing an antioxidant enzyme and cloning of the vector are further contemplated. For example, the presence of enhancers upstream of the promoter or terminators downstream of the coding region, for example, can facilitate expression.

In addition to viral vector-based methods, non-viral expression vectors may also be used to introduce an antioxidant enzyme encoding nucleic acid into a target cell. A review of non-viral methods of gene delivery is provided in Nishikawa and Huang, Human Gene Ther. 12:861-870, 2001. An example of a non-viral gene delivery method for administering an antioxidant enzyme-encoding polynucleotide to a cell is use of a plasmid. Plasmid-based gene delivery methods are generally known in the art. Accordingly, in some embodiments, the antioxidant enzyme expression vector is a plasmid vector.

Additionally, the polynucleotide encoding the antioxidant enzyme gene can be introduced into the target cell by transfecting the target cells using electroporation techniques. Electroporation techniques are well known and can be used to facilitate transfection of cells using plasmid DNA.

Expression vectors that encode an antioxidant enzyme-expressing polynucleotide can be delivered to the target cell in the form of an injectable preparation containing pharmaceutically acceptable carrier, such as saline, as necessary. Other pharmaceutical carriers, formulations and dosages can also be used in accordance with the present invention.

### Ophthalmic Surgical Procedures

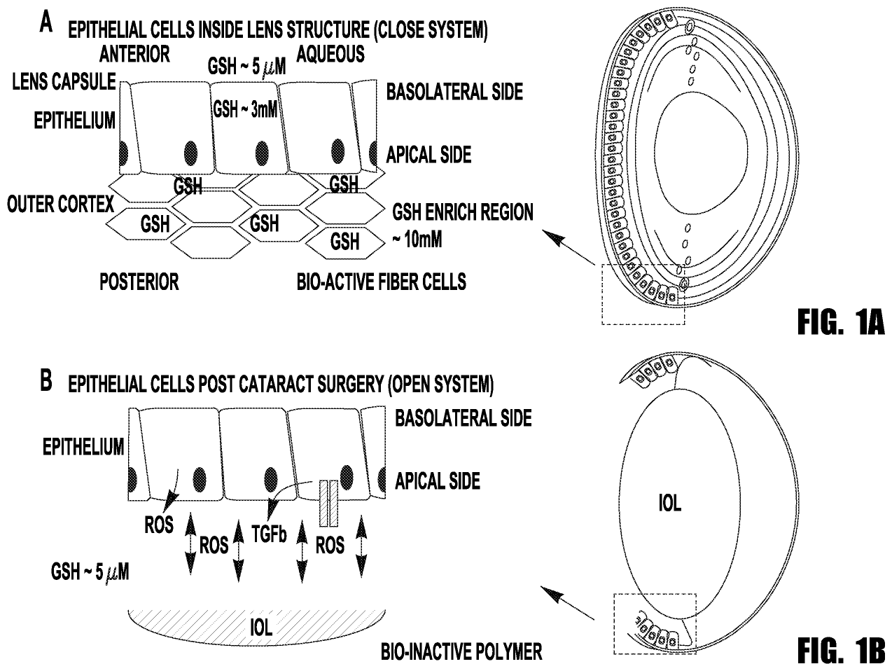

The present invention includes administering a therapeutically effective amount of an antioxidant to a subject undergoing an ophthalmic surgical procedure. Lens epithelial cells face an oxidative stressed growing environment after ophthalmic surgery. During cataract surgery, the lens anterior capsule and entire fiber mass are removed, and an artificial intraocular lens (IOL) is inserted into the eye chamber with posterior capsule still intact. Profound changes have occurred in terms of lens epithelial cell growth environment and microenvironment. As illustrated in FIG. 1, before surgery, the apical side of lens epithelial cells are in contact with the outer fiber cortex, which is a glutathione (GSH) enriched zone with over 10 mM GSH concentration (Giblin, F. J., J. Ocul Pharacol Ther. 16, 121-35 (2000)) (FIG. 1A), the epithelial cells are also surrounded by the lens capsule, a relatively closed environment. GSH is the most abundant endogenous antioxidant, a critical regulator of oxidative stress and cell signaling. At the apical side, any extracellular reactive oxygen species (ROS) accumulation could be easily detoxified by a powerful GSH-enriched zone. In contrast, after ophthalmic surgery, lens epithelial cells are completely separated from lens fiber cells and face a wild open growth environment (FIG. 1B). The GSH concentration in the apical side is reduced over 2000 times to 5 μM levels. Whitson et al., Invest. Ophthalmol Vis Sci 57, 3914-25 (2016). Furthermore, IOL is a biologically inactive lens in that the extracellular ROS generated by lens epithelial cells or surrounding ocular fluid will not be neutralized/detoxified by IOL. As a result, ophthalmic (e.g. cataract) surgery produces a microenvironment that sustains chronic oxidation.

The term “ophthalmic surgical procedure” includes any operation or surgical procedure generally performed on an eye, such as LASIK, orbital surgery cataract surgery, intraocular lens surgery, corneal transplant surgery, glaucoma surgery, or the like. A variety of different ophthalmic surgical procedures are known to those skilled in the art. LASIK, or laser in-situ keratomileusis, uses a laser to correct nearsightedness, farsightedness or astigmatism, by mapping the eye and sculpting the cornea. PRK, or photorefractive keratectomy, also reshapes the cornea, but differs from LASIK in that it uses a cool, ultraviolet laser light on the surface of the cornea. Corneal transplants replace a damaged cornea. Cataract surgery is done to remove a clouded lens of the eye. Glaucoma surgery is used to reduce the intraocular pressure on the eye, while orbital surgery repairs trauma to the eye.

In some embodiments, the ophthalmic surgical procedure is cataract surgery. A cataract is a clouding or development of an opaque area in the lens. Most cataracts form as part of the aging process, but some are associated with congenital or systemic pathological conditions and others are related to ocular trauma. Cataracts are formed by the clumping of the proteins in the lens and the opacification that ensues, which hinders light transmission and normal vision. In cataract surgery, the lens anterior capsule and fiber mass are removed, and an artificial intraocular lens is inserted into the eye chamber with the posterior capsule still intact.

The antioxidant should be administered proximal in time to when the ophthalmic surgical procedure is carried out so the antioxidant can decrease oxidative damage to the eye that occurs as a result of the surgical procedure. The amount of time that is “proximal” can vary, depending on the pharmacokinetics of the antioxidant being used, and the extent and duration of the oxidative damage resulting from the ophthalmic surgical procedure. Proximal in time includes administering the antioxidant during the ophthalmic surgical procedure. However, in some embodiments, the antioxidant is administered before the ophthalmic surgical procedure, while in other embodiments the antioxidant is administered after the ophthalmic surgical procedure. When administered before or after the ophthalmic surgical procedure, proximal in time includes administration within 10 minutes of the surgical procedure, within 30 minutes of the surgical procedure, within 1 hour of the surgical procedure, within 4 hours of the surgical procedure, within 8 hours of the surgical procedure, within 24 hours of the surgical procedure, or in some embodiments within one week of the surgical procedure.

### Administration and Formulation

The administration of an antioxidant proximal in time with the ophthalmic surgical procedure may be performed by known methods, such as the continuous irrigation and aspiration of the solution into the eye during surgery. Alternatively or additionally, the solution may be separately instilled or injected into the anterior chamber through a needle or cannula attached to a syringe at the conclusion of or immediately following the surgical procedure to inflate the globe. Such an injection may also be considered to be irrigation of the anterior chamber. Further, the solution according to the invention may be topically applied as eye drops via a dropper-type bottle or dripped from a syringe onto the external surface of the eye.

The antioxidant can be administered “neat,” without being formulated with other ingredients, or the antioxidant can be administered with a pharmaceutically acceptable carrier. The pharmaceutically acceptable carrier includes one or more inert components (e.g., non-biologically active), non-polymeric components, and/or components which are biocompatible and biodegradable. Additional components that can be included in a pharmaceutically acceptable carrier include, but are not limited to, buffering agents, salts (e.g., physiological saline), organic solvents, particle dispersion stabilizers or suspending agents, thickening agents, surfactants, pharmaceutically acceptable excipients or vehicles, carriers, and diluents (e.g., solutes that render the formulation isotonic with the bodily fluids of the intended recipient). Injectable formulations of the compositions can, in some embodiments, contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. Furthermore, physiologically-acceptable excipients can include, for example, 5% dextrose, 0.9% saline, Ringer's solution, Water-for-Injection, or 5% glucose solution.

Buffering agents of interest for use with the subject compositions include, but are not limited to, organic acid salts such as salts of citric acid, ascorbic acid, gluconic acid, carbonic acid, tartaric acid, succinic acid, acetic acid or phthalic acid; Tris, thomethamine hydrochloride, or phosphate buffer. In addition, amino acid components can also be used as buffering agent. Such amino acid component includes without limitation glycine and histidine. In some cases, a histidine buffer is particularly useful. The subject aqueous compositions include such buffering agent or pH adjusting agent to provide improved pH control. In one embodiment, an aqueous pharmaceutical composition of the invention has a pH between 5.0 and 8.0, between 5.5 and 7.5, between 5.0 and 7.0, between 6.0 and 8.0, between 6.5 and 8.0, or between 7.0 and 8.0. In a specific embodiment, an aqueous composition of the invention has a pH of about 7.4. In a specific embodiment, an aqueous composition of the invention has a pH of about 7.5.

Other contemplated excipients that can be included in a pharmaceutically acceptable carrier include, for example, flavoring agents, antimicrobial agents, bacteriostats, bactericidal antibiotics, antistatic agents, lipids such as phospholipids or fatty acids, steroids such as cholesterol, protein excipients such as serum albumin (human serum albumin), recombinant human albumin, gelatin, casein, salt-forming counterions such as sodium, and the like. These and additional pharmaceutical excipients and/or additives suitable for use in the subject compositions are known in the art, e.g., as listed in The Handbook of Pharmaceutical Excipients, 4th edition, Rowe et al., Eds., American Pharmaceuticals Association (2003); and Remington: the Science and Practice of Pharmacy, 21th edition, Gennaro, Ed., Lippincott Williams & Wilkins (2005).

The antioxidant can be administered to a subject using a variety of different applicators including needles, plastic, ceramic, or metal applicators, and the like. For example, in some embodiments, the antioxidant is administered to a subject by directly injecting the antioxidant composition through a small bore needle, such as a 25 or 27 gauge needle, into the vitreous humor of an eye of a subject. In some embodiments, the composition is administered to a subject by directly injecting the antioxidant composition through a needle, such as a needle having a gauge in the range of 28 to 30 (e.g., 28, 29 or 30 gauge), into the vitreous humor of an eye of a subject.

While it is preferable to administer the antioxidant to the eye, in some embodiments, the antioxidant can be administered to the subject using other forms of administration (e.g., oral or parenteral), so long as the antioxidant has pharmacological characteristics such that systemic administration will result still in an effective amount of the antioxidant reaching the eye of the subject. For example, in some embodiments the antioxidant is administered orally in a suitable form such as a powder, granule, tablet, capsule, suspension, emulsion, syrup, aerosol, etc.

Other contemplated excipients, which may be included in the pharmaceutically acceptable carrier used to administer the antioxidant include, for example, flavoring agents, antimicrobial agents, bacteriostats, bactericidal antibiotics, antistatic agents, lipids such as phospholipids or fatty acids, steroids such as cholesterol, protein excipients such as serum albumin (human serum albumin), recombinant human albumin, gelatin, casein, salt-forming counterions such as sodium, and the like. These and additional pharmaceutical excipients and/or additives suitable for use in the subject compositions are known in the art, e.g., as listed in The Handbook of Pharmaceutical Excipients, 4th edition, Rowe et al., Eds., American Pharmaceuticals Association (2003); and Remington: the Science and Practice of Pharmacy, 21th edition, Gennaro, Ed., Lippincott Williams & Wilkins (2005).

Regardless of the particular mode of administration used in accordance with the methods of the present invention, the antioxidant compositions described herein can be administered in an amount effective to achieve the desired response (e.g., a reduction in oxidative damage). As such, the term “therapeutically effective amount” is used herein to refer to an amount of the therapeutic composition (e.g., an antioxidant composition including a pharmaceutically acceptable carrier) sufficient to produce a measurable biological response (e.g., a reduction in oxidative damage). Actual dosage levels of active ingredient can be varied so as to administer an amount of the antioxidant that is effective to achieve the desired therapeutic response for a particular subject and/or application. The selected dosage level and amount of the antioxidant and the other components of the composition will depend upon a variety of factors including the activity of the antioxidant, formulation, the route of administration, combination with other drugs or treatments, severity of the condition being treated, and the physical condition and prior medical history of the subject being treated. In some cases, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art.

### Intraocular Lens

In some embodiments, the antioxidant is administered from an antioxidant eye lens comprising an intraocular lens (IOL) coated with an antioxidant that is implanted in the eye of the subject. IOLs have been developed and inserted into various locations of the eye and can be used to supplement or correct the vision provided by the natural crystalline lens of the eye or can replace the natural crystalline lens of the eye. Lenses that supplement or correct the vision without replacing the natural crystalline lens are typically referred to as Phakic Lenses while lenses that replace the natural crystalline lens are typically referred to as Aphakic lenses. Phakic lenses can be located within the anterior chamber (AC) of the eye (AC Phakic lenses) or the posterior chamber (PC) of the eye (PC Phakic Lenses).

In some embodiments, the ophthalmic material of the IOL is suitable for loading with the antioxidant that is to be delivered to the eye. The ophthalmic material can be part of the IOL itself, or it can be included as a drug-eluting coating for the IOL. The ophthalmic material is typically relatively hydrophobic and, preferably, the antioxidant also exhibits a degree hydrophobicity. In one preferred embodiment, the material is provided as an Aphakic IOL and the therapeutic agent includes a substantial amount of antioxidant for reducing oxidation that may be present after cataract surgery.

The ophthalmic material is preferably a polymeric material that is comprised of a hydrophobic component, a hydrophilic component or both. In some embodiments, the ophthalmic material is an acrylic, which, as used herein, means that that the material includes as least one acrylate. See US Patent Publication No. 2017/0319474. The ophthalmic material can additionally include a variety of further ingredients, such as flexibilizers, UV absorbers, polymerization agents, curing and/or cross-linking agents, combinations thereof or the like.

In some embodiments, the ophthalmic material is a hydrogel. For example, the ophthalmic material can be either ionic or non-ionic hydrogels containing between 10% and 90%, e.g., 24% or 37.5% to 65% or 75%, water by weight and can have any base curve, e.g., from 8.0 to 9.0. Exemplary hydrogel ophthalmic materials include etafilcon A, vifilcon A, lidofilcon A, polymacon B, vasurfilcon A, and a tetrapolymer of hydroxymethylmethacrylate, ethylene glycol, dimethylmethacrylate, and methacrylic acid. Other suitable hydrogel materials are known to those skilled in the art. The hydrogels may be insoluble or may dissolve over time in vivo, e.g., over one day or one week. The drug is passively delivered, for example, by diffusion out of the hydrogel, by desorption from the hydrogel, or by release as the hydrogel dissolves.

### Kits

Another aspect of the present invention provides kits that find use in practicing the methods of preventing eye oxidation. In some embodiments, the kits for practicing the subject methods include one or more ophthalmic compositions, which include one or more antioxidants, e.g., as described herein. As such, in certain embodiments the kits may include a single ophthalmic composition, present as one or more unit dosages.

Any of the antioxidants or other components described herein may be provided in the kits. A variety of components suitable for use in practicing the methods of decreasing eye oxidation may find use in the kits. Kits may also include one or more components including, but not limited to, means for intra-ocular (e.g., intravitreal) injection (e.g., an injection device suitable for intravitreal injection), an eye numbing agent, a sterile dilution buffer, a trocar device, means for measuring intraocular pressure, a sealed package configured to maintain the sterility of the ophthalmic pharmaceutical composition, sterile containers, pharmaceutically acceptable solutions, freeze-dried solids thereof, tubes, buffers, etc., and instructions for use.

The antioxidant and a pharmaceutically acceptable carrier (i.e., ophthalmic composition) can be pre-loaded into an injection device (e.g., a syringe) suitable for injection, or can be included in the kit as a separate component. In certain instances, the ophthalmic composition included in the kit consists essentially of an antioxidant in a pharmaceutically acceptable carrier. In certain embodiments of the kit, the pharmaceutically acceptable carrier comprises a non-ionic surfactant. In certain embodiments of the kit, the pharmaceutically acceptable carrier comprises a sterile biocompatible buffer. Any of the antioxidants described herein can be utilized. In certain embodiments of the kit, the antioxidant is N-acetyl cysteine or glutathione ethyl ester.

The various components of the kits may be present in separate containers, or some or all of them may be pre-combined into a mixture in a single container, as desired. In certain embodiments, the kit includes a sterile container containing a pharmaceutically acceptable solution of the subject composition; and an optionally sealed package configured to maintain the sterility of the sterile container.

In addition to the above components, the subject kits may further include (in certain embodiments) instructions for practicing the methods of decreasing eye oxidation. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), hard drive etc., on which the information has been recorded. Yet another form of these instructions that may be present is a website address which may be used via the internet to access the information at a removed site.

The present invention is illustrated by the following example. It is to be understood that the particular example, materials, amounts, and procedures are to be interpreted broadly in accordance with the scope and spirit of the invention as set forth herein.

### Example

### Chronic Oxidative Stress Promotes Epithelial-Mesenchymal Transition in the Lends Via a Wnt/β-Catenin-Mediated TGFβ Independent Pathway: Relevance for Cataract Therapy

The inventors used cell culture, chronic oxidative stress mouse models, and mouse mock cataract surgery models to address the role of chronic oxidation in the pathogenesis of lens epithelial cell fibrosis via EMT and its signaling pathways.

### Results

EMT marker expression is enhanced in the lenses of LEGSKO and Gclm KO mice.

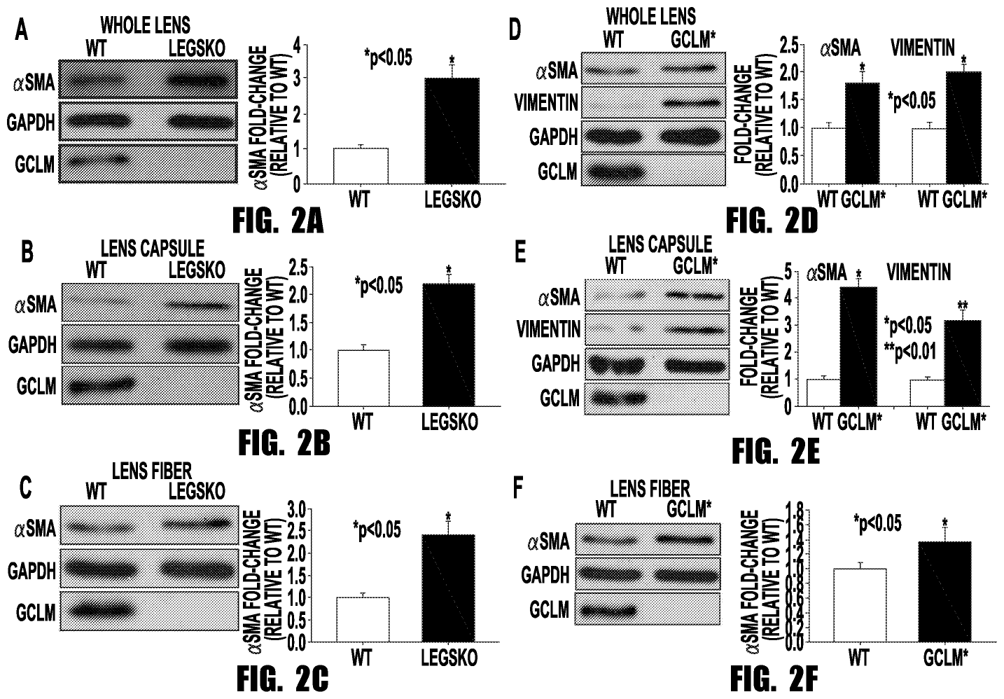

Two genetically engineered mouse models targeting GSH de novo synthesis were tested. The lens conditional γ-glutamylcysteine ligase, catalytic subunit (Gclc) knockout (Gclc^{-/-}, LEGSKO) mouse had more than 50% suppressed GSH levels (1.0-1.5 mM) compared to wild type (WT) (3-5 mM). Fan et al., PLoS One 7, e50832 (2012). The somatic γ-glutamylcysteine ligase, modifier subunit (Gclm) knockout mouse (Gclm^{-/-}) had over 70% suppression of lens GSH levels (0.8-1.2 mM) vs. WT lenses, based on analysis using LC/MS. Whitson et al., Invest Ophthalmol Vis Sci 57, 3914-3925 (2016). All mice used were in C57BL/6 genetic background and age-matched at four months. Whole lens, lens capsule (with adherent lens epithelia), and lens fiber sections were used to test various mesenchymal-like marker expressions. As illustrated in FIGS. 2A & 2D, a more than three-fold and two-fold increase of αSMA expression was seen by immunoblot analysis in the whole lens protein extract of Gclc^{-/-} and Gclm^{-/-} compared to WT, respectively. Over two-fold and four-fold increases of lens capsule αSMA expression was seen in Gclc^{-/-} and Gclm^{-/-} mice compared to WT, respectively. Similarly, significantly increased vimentin was found in both whole lens and lens capsule of the Gclm^{-/-} mouse compared to WT. Furthermore, αSMA was also significantly increased in lens fiber cells homogenate in both Gclc^{-/-} and Gclm^{-/-} mice compared to WT (FIGS. 2C and 2F).

Blocking intracellular GSH biosynthesis prompts EMT marker expression and antioxidants attenuate such activation in human and porcine lens epithelial cells.

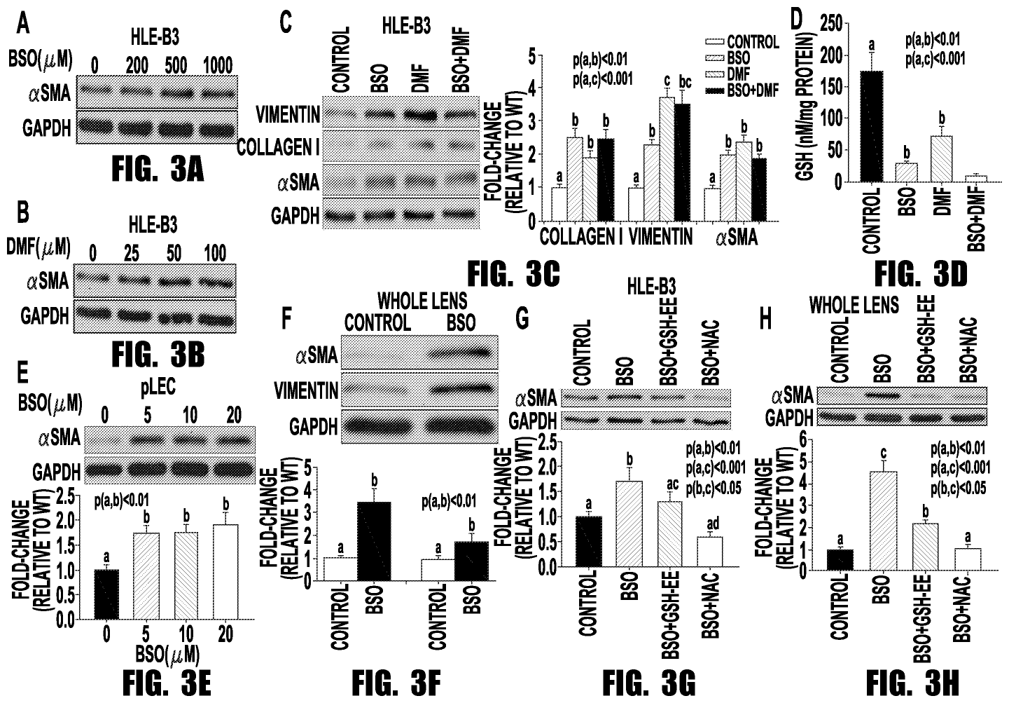

To test whether the above findings from mouse experimental models also occur in cell culture systems, a human lens epithelial cell line (HLE-B3) and primary cultured porcine lens epithelial cells (pLEC) with or without buthionine sulfoximine (BSO), a γ-glutamyl cysteine ligase (GCL) inhibitor, or dimethyl fumarate (DMF), were treated to deplete GSH. As shown in FIGS. 3A&B, αSMA expression was significantly upregulated after 24 hr treatment with increasing concentration of BSO and DMF. Some degree of dose response up to 500 μM BSO and 50 μM DMF was noted. However, further increasing the concentration of BSO or DMF had only moderate effects on αSMA expression. The inventors used 500 μM BSO and 25 μM DMF conditions for the rest of the HLE-B3 studies, and the intracellular GSH concentration with or without treatment is shown in FIG. 3D. As shown in FIG. 3C, type I collagen and vimentin were all significantly elevated in HLE-B3 cells after a 24 h challenge with BSO, DMF, or BSO combined with DMF. Interestingly, a 100 times lower concentration of BSO (5 μM) was required to stimulate a similar fold of αSMA production in primary porcine lens epithelial cells in 24 hr (FIG. 3E). Higher dosages of BSO (>100 μM) were found to trigger massive cell death and detachment, indicating cell apoptosis rather than transformation. In addition, αSMA and vimentin production was tested in mouse lens ex vivo culture. As shown in FIG. 3F, an over 3.5-fold increase of αSMA and ˜2-fold increase of vimentin expression was seen in lenses treated with 500 μM BSO for 48 hrs compared to medium alone. These results indicate that moderate oxidative stress regulates intracellular gene expression and cell transformation while an overdose of oxidative stress triggers cell death.

Inversely, the inventors attempted to rescue cell transformation by antioxidant treatment. As illustrated in FIGS. 3G and 3H, αSMA production in HLE-B3 cells and ex vivo cultured mouse lenses after BSO stimulation was significantly attenuated when cells or lenses were co-treated with either 10 mM N-acetyl cysteine (NAC) or 0.5 mM glutathione ethyl ester (GSH-EE). In particular, 10 mM NAC could completely abolish the BSO-induced αSMA production in both HLE-B3 cells and lens ex vivo culture models, and GSH-EE could partially, but significantly, block αSMA production (FIGS. 3G&H).

Oxidative stress enhances TGFβ-mediated EMT signaling.

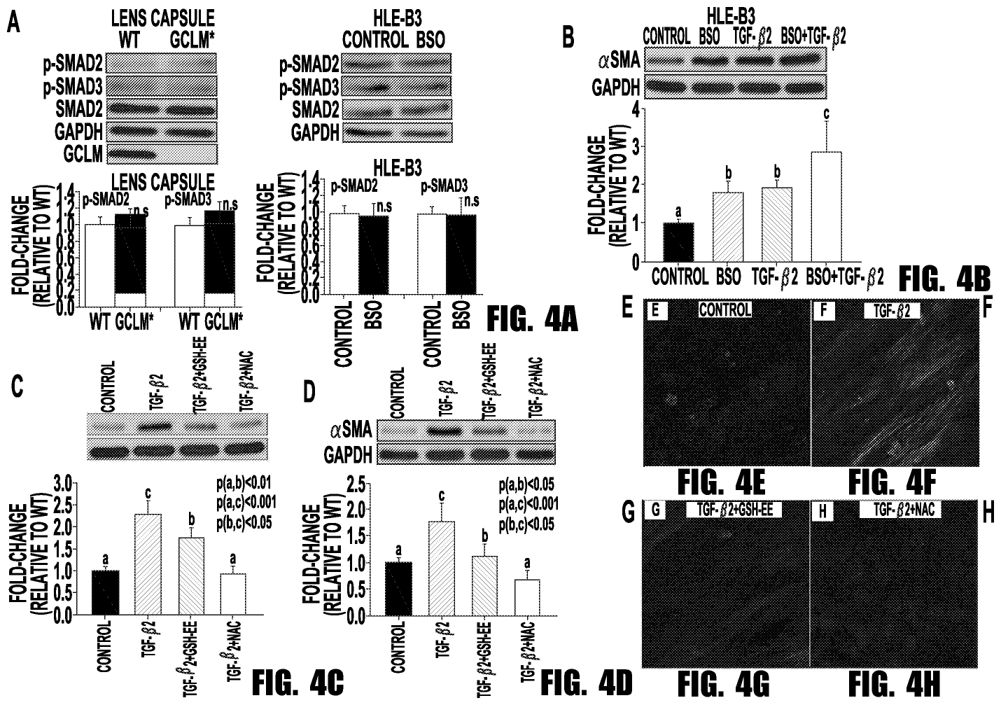

Given the fact that TGFβ is a very potent profibogentic cytokine in EMT signaling, the inventors wanted to clarify whether the higher mesenchymal-like marker protein production is the consequence of higher TGFβ levels in Gclc^{-/-} or Gclm^{-/-} mouse, particularly in the aqueous humor where the major isoform, TGFβ2, directs lens epithelial cell signaling transduction, as has been reported in previous studies. Zhu et al., J Cataract Refract Surg, 42, 232-238 (2016); Matthaei et al., Invest Ophthalmol Vis Sci 56, 2749-2754 (2015) Both total and active TGFβ2 was determined in aqueous humor by ELISA. As shown in Table 1, there were no noticeable changes in the total or active form concentration of TGFβ2 in the aqueous humor between Gclc or Gclm knockout mice and WT. In addition, the phosphorylation status of Smad2 and Smad3, a key event in the canonical TGFβ pathway, showed no remarkable changes in the lens capsules of Gclm KO mice compared to WT, or in HLE-B3 cells with or without BSO stimulation (FIG. 4A). These results indicate up-regulation of mesenchymal-like markers is due to decreased GSH content in the lens rather than a TGFβ2 mediated mechanism.

TABLE 1
The total and active TGFβ2 in aqueous humor

| TGFβ2 (pg/ml) | WT | LEGSKO | Gclm^{-/-} |
|---|---|---|---|
| Total | 1012.3 (±47.8) | 1007.9 (±72.8) | 997.91 (±107.2) |
| Active | 5.21 (±2.43) | 6.06 (±3.22) | 4.17 (±2.71) |

To further define the relationship between ROS and TGFβ in EMT signaling, the production of the EMT marker αSMA was tested in HLE-B3 cells after treatment with 500 μM BSO, 1 ng/ml TGFβ2, or 500 μM BSO combined with 1 ng/ml TGFβ2 for 24 hr. As shown in FIG. 4B, blocking GSH biosynthesis by BSO has a synergistic effect with TGFβ2 in αSMA production. However, surprisingly, co-incubation of the antioxidants NAC or GSH-EE could significantly alleviate TGFβ2-mediated αSMA production in the ex vivo lens culture (48 hr challenge) and HLE-B3 cells (24 hr challenge) culture experimental models (FIGS. 4C&D). In particular, 10 mM NAC could completely block αSMA production induced by TGFβ2 (FIGS. 4C&D). This was further confirmed in mouse explant culture models, an ex vivo PCO mimetic model that has been well used in PCO studies. Korol et al., Am J Pathol 184, 2001-2012 (2014) As illustrated in FIG. 4E-H, profound elevation of αSMA was detected in lens explant after 48 hr stimulation by 1 ng/ml TGFβ2 (FIG. 4E&F), and such an increase was completely alleviated by 10 mM NAC (FIG. 4H) and also partially, but significantly, attenuated by 0.5 mM GSH-EE (FIG. 4G).

Wnt/β-catenin pathway is activated in ROS-mediated EMT.

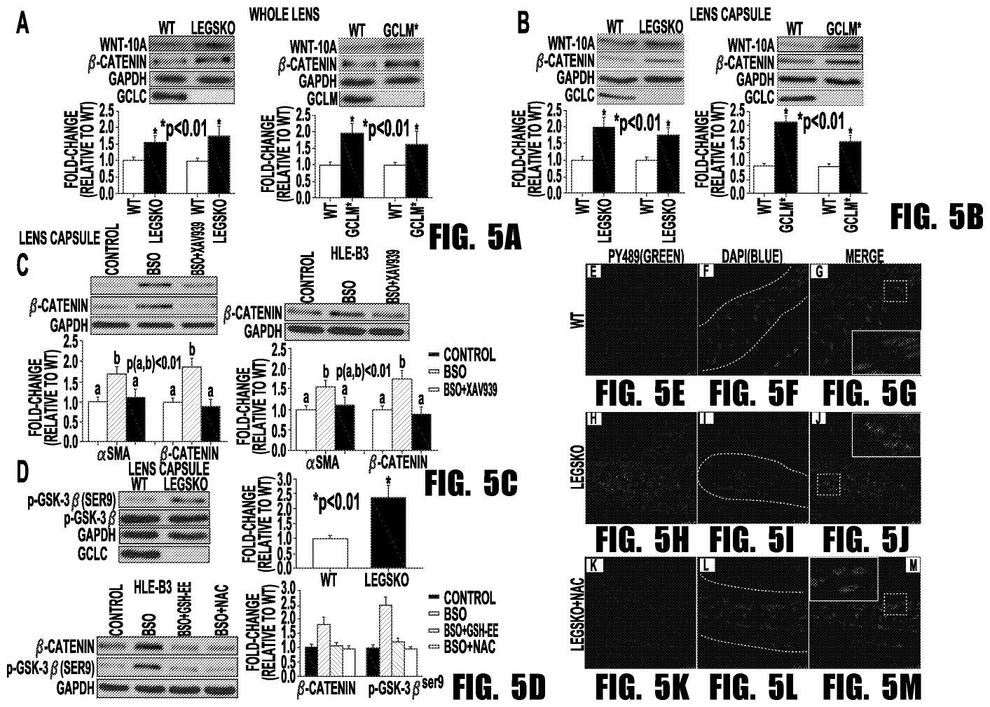

TGFβ and its canonical signaling pathway in ROS-mediated EMT were excluded. The transcriptome profile and pathway analysis from the inventors separate study on lens gene adaptation under decreased concentration of GSH by RNA-seq indicates that the Wnt/β-catenin pathway was the major upregulated pathway associated with EMT signaling. Whitson et al., Invest Ophthalmol Vis Sci., 58(5):2666-2684 (2017) The immunoblot below indicates that both Wnt10a and β-catenin protein levels are highly elevated in the extract of whole lens (FIG. 5A), lens capsule (FIG. 5B), and lens fiber of both Gclc^{-/-} and Gclm^{-/-} mice compared to WT. XAV939, a Wnt inhibitor was able to block both αSMA and β-catenin production in HLE-B3 cells after being challenged by BSO for 24 hr and lens capsule after ex vivo lens culture with or without BSO and BSO plus XAV939 for 48 hr (FIG. 5C).

To further dissect the Wnt/β-catenin signal transduction pathway, the phosphorylation/inactivation of glycogen synthase kinase 3beta (GSK-3β), a key event in Wnt/β-catenin signaling that affects the assembling of β-catenin destruction complex, β-catenin release and translocation to the nucleus was targeted. As illustrated in FIG. 5D, phosphorylation at serine 9 (Ser9), which inactivates GSK-313, was significantly increased in HLE-B3 cells after treatment with BSO for 24 hr and in lens capsules after whole lens ex vivo culture with BSO in the medium for 48 hr. The increased β-catenin expression and Ser9 phosphorylation in GSK-33 were both alleviated when cells were co-treated with 0.5 mM GSH-EE or 10 mM NAC (FIG. 5D). To verify whether Wnt/β-catenin signaling was activated in lens epithelial cells in vivo, mock cataract surgery was performed by removing the lens fiber mass, as reported in previous studies. Lois et al., Arch Ophthalmol 123, 71-77 (2005) The translocation of β-catenin and co-localization with the cell nucleus was visualized by immunofluorescence 72 hrs after the surgical procedure. Bilateral eye surgery was performed, whereby the left eye served as control while 25l1 of 10 mM NAC was injected into the right eye chamber after surgery followed by continuous topical application of 10 mM NAC eye drop every 6 hours from 7 am to 11 pm postoperatively. As illustrated from FIG. 4E-M, robust nuclear colocalization of β-catenin could be seen in Gclc^{-/-} mice 72 hr after surgery and this colocalization signal was significantly attenuated in NAC treated eyes.

Impaired GSH biosynthesis promotes EMT signaling in vivo.

To further clarify the role of oxidative stress in the EMT process in vivo, mock cataract surgery was performed on mice by removing the lens fiber mass without damage to the posterior capsule, similar to human cataract surgery but without insertion of an intraocular lens implant. As described, the right eye was used for antioxidant treatment and the left eye served as control. The tissue collected immediately after surgery was used as a time zero control. Histology at postoperative day 5 visualized by Hematoxylin and Eosin (H&E) showed that Type I collagen and αSMA demonstrated remarkable increase in LEGSKO mice compared to WT from 48 hr to 120 hr postoperatively, while barely detectable signal was observed in the time zero control. The inventors did pick up higher signals of these EMT markers at time zero in LEGSKO and Gclm KO mice compared to WT when the confocal capture sensitivity was adjusted properly. Massive cell accumulation was seen in the posterior capsule at 96 hr and 120 hr after surgical procedure in both WT and LEGSKO mice, but with significant intensity of both collagen I and αSMA in LEGSKO mice compared to WT. Similarly, fibronectin and vimentin also demonstrated significantly increased expression in LEGSKO mice compared to WT. The 10 mM NAC injection immediately after surgery and continuous topical application demonstrated remarkable attenuation of increased fibronectin/vimentin and collagen I/αSMA expression compared to control eyes in both WT and LEGSKO mice. As expected, Gclm KO mice also demonstrated profound increases of fibronectin, vimentin, collagen I, and αSMA compared to WT, and 2 mM GSH-EE could partially but significantly attenuate the production of these EMT marker proteins.

### DISCUSSION

TGFβ is an important player in lens epithelial cell fibrosis and PCO formation. However, surprisingly, despite the strong findings from in vitro and ex vivo capsular bag culture experimental models that EMT signaling and PCO formation could be attenuated via blocking TGFβ-mediated pathways (Eldred et al., Invest Ophthalmol Vis Sci 53, 4085-4098 (2012)), no effect was found in an in vivo rodent PCO model when anti-TGFβ2 antibody intervention was tested. Lois et al., Invest Ophthalmol Vis Sci 46, 4260-4266 (2005) Furthermore, PCO is a chronic disease that usually develops a few years after the surgical procedure. The remnant lens epithelial cells have to go through the acute phase immediately after a traumatic injury caused by the surgical procedure, which is followed by transition to the subacute and chronic phase after the initial wound healing process is over. Inflammatory cytokines and growth factors are produced which reach their peak levels during the acute phase and then continuously decline as the healing stage ends. Kawai et al., Invest Ophthalmol Vis Sci 53, 7951-7960 (2012) This is echoed by the short- and long-term clinical observation of lens epithelial cell grouping behavior, so called Elschnig pearls, which indicate that the number and size of Elschnig pearls increase significantly 2 weeks postoperatively but start to decrease or disappear at 4 months postoperatively. Buehl et al., J Cataract Refract Surg 31, 962-968 (2005); Buehl et al., J Cataract Refract Surg 31, 2120-2128 (2005) Elschnig pearls' number and size reoccurred at postoperative year 1-3, especially in those patients with PCO complications. These studies suggest that there are unknown processes that continuously drive lens epithelial cell proliferation, migration and transdifferentiation during the chronic phase.

Lens epithelial cells are surrounded by a capsule at their basolateral side while their apical side is in contact with lens fiber cells. Thus, overall, lens epithelial cells are in a relatively “closed” growth environment. During cataract surgery, the fiber mass is removed and the lens epithelial cells are separated from lens fiber and either in direct contact with an intraocular lens (IOL) or with aqueous humor, thus a relatively “open” growth environment. Moreover, the lens' outer fiber layer is a GSH enriched zone with over 10 mM concentration (Giblin, F. J., J Ocul Pharmacol Ther 16, 121-135 (2000)), while the aqueous humor only has ˜5 μM GSH based on our recent study. Whitson et al., Invest Ophthalmol Vis Sci 57, 3914-3925 (2016) As a result, profound changes occur after cataract surgery, especially in the extracellular GSH content of lens epithelial cells. The inventors hypothesized, and suggest in this work, that, postoperatively, elevated oxidative stress also becomes part of the lens epithelial cell growth environment and that this profoundly impacts the biology of the surviving lens epithelial cells. In particular, the biologically inactive IOL is unable to neutralize or detoxify extracellular ROS generated by lens epithelial cells or surrounding ocular fluid.

ROS have been implicated as playing a pivotal role in EMT signaling and tissue fibrosis either acting alone or by modulating TGFβ-mediated signaling. On one hand, TGFβ can increase intracellular ROS formation by either increasing NADPH oxidase production or impairing the intracellular antioxidant defense system, i.e. inhibiting GSH biosynthesis by GCL. Liu et al., Free Radic Biol Med 53, 554-563 (2012) On the other hand, ROS could often induce either TGFβ production or activate TGFβ from its latent state. Barcellos-Hoff, M. H., and Dix, T. A., Mol Endocrinol 10, 1077-1083 (1996) Interestingly, this work suggests that decreased intracellular GSH concentration has a synergistic effect in EMT signaling with TGFβ2. However, the complete blockage of TGFβ2-induced αSMA production in lens epithelial cells and the lens explant culture model by 10 mM NAC suggests that there is crosstalk between ROS and TGFβ in EMT signaling. At the same time, it also suggests that ROS play an important role in EMT signaling and tissue fibrosis.

The inventors found that blocking intracellular GSH biosynthesis could trigger EMT signaling and EMT marker production based on two GSH biosynthesis deficiency mouse models, as well as a human lens epithelial cell line and primary porcine lens epithelial cells after either BSO or DMF stimulation to reduce intracellular GSH levels. Equally important, co-incubation with an antioxidant, such as GSH-EE or NAC, could attenuate EMT marker production in a significant manner. Surprisingly, it was found that no remarkable changes in the total and active TGFβ2 levels in the aqueous humor nor in phosphorylation status of Smad2/3, a key event in canonical TGFβ signaling, in Gclc and Gclm KO lenses compared to WT. This suggests that EMT signaling induced by decreased intracellular GSH levels is not mediated by extra TGFβ2 or activated TGFβ2 production, a major TGFβ isoform in lens epithelial cells. Saika et al., Graefes Arch Clin Exp Ophthalmol 238, 283-293 (2000)

Further study suggested that the EMT signaling modulated by decreased intracellular GSH concentrations is mediated via the Wnt/β-catenin signaling pathway. Over a 20-fold up-regulation of Wnt10a was found in our recent RNA-seq study when comparing lens epithelial cells' gene transcriptome profile between LEGSKO and WT mice. Whitson et al., Invest Ophthalmol Vis Sci., 58(5):2666-2684 (2017) The inventors saw an over 2-fold Wnt10a protein expression in the lens capsule. The discrepancy between mRNA and protein up-regulation may be due to Wnt10a secretion into the extracellular space, i.e. aqueous humor, because Wnt protein secretion is a common mechanism for its signal transduction. Mikels, A. J., and Nusse, R., Oncogene 25, 7461-7468 (2006) Activation of the Wnt and Frizzled receptor complex subsequently disrupts the AP/Axin/GSK-3β complex, triggering phosphorylation and inactivation of GSK-3β, i.e. serine 9 phosphorylation, which in turn stabilizes β-catenin, allowing β-catenin nuclear translocation and target gene regulation. Wu, D., and Pan, W., Trends Biochem Sci 35, 161-168 (2010) The inventors found serine 9 phosphorylation in GSK-3β was significantly increased in the mouse lens capsule when GSH biosynthesis is impaired, and when human lens epithelial cells are being challenged by BSO to block intracellular GSH synthesis. Furthermore, that the phosphorylation of serine 9 could be completely abolished when co-treating the cells with 10 mM NAC, suggests that the Wnt/β-catenin-mediated EMT signaling is oxidative stress dependent. Oxidative stress, with production of ROS and reactive nitrogen species (RNS), is not only limited to toxic effect but also functions as intracellular messengers, regulating gene expression and cell adaptation in both physiological and pathological conditions. The pioneering work from Funato et al. (Nat Cell Biol 8, 501-508 (2006)) suggests that ROS can modulate signaling by the Wnt/β-catenin pathway and, later on, Parola et al. (Cannito et al., Carcinogenesis 29, 2267-2278 (2008)) demonstrated that hypoxia-induced mitochondria ROS production could activate Wnt/β-catenin signaling pathway via phosphorylation/inactivation of GSK-3, which is in agreement with the results described herein.

Importantly, the mouse cataract surgery model tested in this study demonstrates the pivotal role that oxidative stress plays in lens epithelial cell transformation and PCO formation. The remarkable elevation of expression of various EMT markers, such as type I collagen, vimentin, fibronectin and αSMA, in both Gclc and Gclm knockout mice compared to WT, strongly suggest that oxidation could modulate intracellular gene expression and cell adaptation. In this case, oxidative stress produced by decreased intracellular GSH concentration promotes cell transformation rather than apoptosis. Intriguingly, both NAC and GSH-EE could significantly attenuate EMT process by blocking EMT signaling. Interestingly, NAC had a much higher inhibitory effect than GSH-EE in both cell culture and in vivo cataract surgery models, suggesting that the sulfhydryl group of these compounds alone is sufficient to attenuate the redox status that regulates Wnt/β-catenin-mediated EMT signaling. In addition, postoperative β-catenin nuclear translocation in both WT and GSH biosynthesis deficiency mouse models was observed, which supports the hypothesis that EMT signaling modulated by moderate oxidative stress is mediated by a Wnt/β-catenin signaling cascade.

In summary, the present study demonstrates that moderate oxidative stress can modulate lens epithelial cell transformation via Wnt/β-catenin mediated TGF beta independent EMT signaling both in vitro and in vivo. This study suggests that antioxidant intervention is a potential therapeutic approach to treat PCO formation.

### Experimental Procedures

Reproducibility—All experiments shown in figures, unless mentioned specifically, consist of at least five biological replicates that are reproducible.

Reagents—All chemicals used were of analytical reagent grade. Milli-Q water was used for preparation of standards and reagents. Human TGFβ2, N-acetyl cysteine, glutathione, glutathione ethyl ester, XAV939, buthionine sulfoximine and dimethyl fumarate and all other sodium salt and chemical reagents were all purchased from Sigma-Aldrich (St. Louis, Mo.).

Animals—All animal experiments were conducted in accordance with procedures approved by the Case Western Reserve University Animal Care Committee and conformed to the ARVO Statement for use of Animals in Ophthalmic and Vision Research. Animals were housed under diurnal lighting condition and allowed free access to food and water. The lens conditional gamma glutamyl-cysteine ligase catalytic subunit knockout mice (Gclc), named as LEGSKO mice, were created by our group as has been described in our previous report. Fan et al., PLoS One 7, e50832 (2012) The systemic gamma glutamyl-cysteine ligase modifier subunit knockout mice (Gclm^{-/-}) was kindly provided by Terrence J. Kavanagh, University of Washington. Cole et al., J Toxicol., 2011, 157687 (2011)

Cell Culture—Human lens epithelial cells (HLE-B3) were grown in DMEM medium with 10% fetal bovine serum (FBS), 2 mM glutamine and 50 unit/ml penicillin streptomycin (ThermoFisher, Waltham, Mass.) at 37° C. in a humidified 5% CO₂ incubator. For primary porcine lens epithelial cell culture, pig eyes were collected at a local slaughterhouse within two hours of postmortem time, placed on ice and transported to the lab (about 1 hr) for immediate processing. First, the isolated lens was incubated with 2 ml of 0.25% trypsin at 37° C. for 2 min to remove any attached non-lens cells. Then, after a brief rinse with HBSS, the lens capsule was isolated and rinsed three times with HBSS before incubation with 2 ml of 0.5% trypsin for 5 min. 5 ml of complete DMEM medium was added and tissue was disrupted by pipetting up and down 10-15 times with a pipette fitted with a 1000 μl tip. The tissue homogenate was then passed through a 100 μm cell strainer. The cells were collected by low speed centrifugation at 150×g and placed in a 35 mm laminin-coated tissue culture dish with DMEM medium supplied with 10% FBS, 2 mM glutamine and 50 unit/ml penicillin streptomycin (ThernoFisher). The cells were cultured at 37° C. in a humidified incubator with 5% CO₂.

Lens Ex Vivo Culture and Lens Explant Culture.

The isolated intact lens was rinsed three times with HBSS and then cultured in 12-well-plates with serum-free Medium 199 (ThermoFisher) with 50 unit/ml penicillin streptomycin at 37° C. in 5% CO₂ incubator, as described above. The lens explant was cultured following the same procedure as described by a previous report. Tamiya et al., Exp Eye Res 71, 591-597 (2000). In brief, the mouse lens was placed in an anterior-toposterior orientation with the anterior attached to the culture dish. The lens posterior capsule was torn/cut by the tip of extra-fine forceps and then peeled towards anterior capsule and pinned on the plastic culture dish. The lens fiber mass was removed thereafter. The explant was cultured in serum-free MEM medium supplied with 50 unit/ml penicillin streptomycin at 37° C. in 5% CO₂ incubator as described above. Only healthy growing explants were used for the described study and any unhealthy looking explants, such as folded explants or those with cell detachment, were discarded.

Treatment Procedures.

Cells were placed 24 hr before the treatment and fresh medium was replaced immediately before the treatment. The indicated concentrations of BSO, DMF, NAC, GSH-EE or TGFβ2 were added to fresh medium and incubated at various time intervals as indicated. After treatment time, the cells were harvested after three washes with ice-cold PBS and subjected to immunoblot or other assays as described below. For whole lens culture, the serum-free medium with or without BSO, NAC, GSH-EE or TGFβ2 was incubated immediately after isolation for 48 hr and the whole lens or lens capsule was collected for further analysis. For lens explant culture, 48 hr after explant culture, various stimulates were used in fresh serum-free medium for an additional 48 hr, and the explant was fixed with 10% formaldehyde for 10 min before immunofluorescence stain, as described below.

Mouse Mock Cataract Surgery.

Age-matched four month old mice were used in this study. Mice were anesthetized and a central corneal incision was made. The lens fiber mass was removed by hydrodissection and the remaining lens was carefully washed by HBSS to remove any lens fiber debris. Saline buffer was injected to help mice to restore their proper eye shape. Eye drops containing the antibiotic Ofloxacin were applied once immediately after surgery, and mice were sacrificed at 2, 3, 4 and 5 days after surgery. For each mouse, bilateral surgeries were performed and 25 μl of 10 mM NAC or 2 mM of GSH-EE was injected into right eye chamber immediately after surgery and continual eye drop topical application was given every 6 hr from 7 am to 11 pm. The untreated left eye served as a control. Eyes collected immediately after surgery served as time zero control. At least 6 mice were used in each group. At each time point, eyes were carefully dissected and placed in 4% paraformaldehyde for 18 hr, and then subjected to paraffin embedding and sectioning.

Immunofluorescence.

For paraffin embedded slides, 6 μm thick sections were deparaffinized and rehydrated using standard procedure. The slides were rinsed with PBS for 10 min and antigen retrieval was carried out in 0.1M sodium citrate, pH 6.0 buffer in a microwave oven using a 4 min at power 5 plus 8 min at power 3 program. The slides were then blocked and permeabilized with 5% normal goat serum, 0.3% triton X-100 for 30 min. The primary antibody, rabbit polyclonal collagen I antibody (1:250) (Abcam, Cambridge, Mass.), rabbit polyclonal vimentin (1:500) (ThermoFisher), mouse monoclonal fibronectin antibody (1:5) (Developmental Studies Hybridoma Bank, DSHB, University of Iowa), mouse monoclonal active β-catenin (Tyr489) (1:10) (DSHB) or FITC labeled mouse monoclonal αSMA antibody (1:1000) (Sigma-Aldrich) were applied in 0.1% BSA PBS solution and incubated at 4° C. overnight in a humid chamber. After wash, the Alexa-488 or/and Alexa-594 conjugated secondary antibodies (ThermoFisher) were then applied and incubated at room temperature for one hour in a humid chamber. After wash, the slides were mounted with Prolong antifade with DAPI (ThermoFisher). For lens explant culture, the explant was briefly fixed with 10% formaldehyde for 10 min and then subjected to directly staining procedure without antigen retrieval procedure.

Confocal microscope—All confocal images were collected by a Leica SP8 gSTED confocal microscope equipped with two 3 Hyd SP GaAsP detectors and the AOBS system lasers include a 405 nm, Argon (458, 476, 488, 496, 514 nm), a tunable white light (470 to 670 nm), and a 592 nm STED depletion laser. For proper comparison, each channel of fluorescence was adjusted to the highest sensitivity/intensity but without over saturation from samples with the strongest signal. In our case, Gclc and Gclm KO mice samples were used for such purpose. The parameters were then used for all sample image collection. At least 10 images were captured at each sample.

Glutathione Assay.

Intracellular GSH was determined by the LC/MS method described in our previous study. Whitson et al., 2016, ibid. In brief, the cells were washed three times with ice-cold Ca²⁺/Mg²⁺ free PBS, harvested via cell scraper method, and transferred to a pre-chilled 1.5 ml pestle tube (Kimble) with 250l1 ice-cold extraction buffer, 0.1% Triton X-100, 0.6% sulfosaliclic acid. The cells were homogenized by pestle for 1 min and then sonicated for 2 min under ice-water bath. The supernatant after 10 min centrifugation at 25,000 g was collected and formic acid was added to the final concentration of 0.1% and then subjected to LC/MS analysis. The GSH was calculated as nmol/mg protein.

Immunoblot Assay.

The cells, whole lens, lens capsule, and lens fibers were collected and lysed in lysis buffer containing 20 mM Tris pH 7.5, 1 mM EDTA, 1 mM EGTA, 150 mM NaCl, 1% Triton X-100, 2.5 mM sodium pyrophosphate, 1 mM bα-Glycerolphosphate, 1 mM Na₃VO₄, 1 mg/ml Leupeptin, 1 mM PMSF on ice for 10 min. The protein concentration from the supernatant was determined by protein BCA assay (ThermoFisher). The protein extract was further processed for immunoblot analysis and probed for αSMA, collagen I, vimentin, phosphorylated Smad2, phosphorylated Smad3, phosphorylated GSK-3β and (β-catenin. All data were normalized to the level of GAPDH.

Statistical Methods.

All values were expressed as means±SE. Statistical analysis was performed according to methods previously described in detail. Sell et al., FASEB journal, 14, 145-156 (2000) In brief, student t test and one-way ANOVA Test were computed using SPSS software. Significance was considered P<0.05.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of decreasing eye oxidation, comprising administering a therapeutically effective amount of an antioxidant to a subject undergoing an ophthalmic surgical procedure.

2. The method of claim 1, wherein the antioxidant is a small molecule antioxidant.

3. The method of claim 2, wherein the antioxidant is thiol-based antioxidant.

4. The method of claim 3, wherein the thiol-based antioxidant is N-acetyl cysteine or glutathione ethyl ester.

5. The method of claim 1, wherein the antioxidant is an antioxidant enzyme activator.

6. The method of claim 5, wherein the antioxidant enzyme activator is a polynucleotide.

7. The method of claim 6, wherein the polynucleotide is administered using a viral vector.

8. The method of claim 5, wherein the antioxidant enzyme is superoxide dismutase.

9. The method of claim 1, wherein the ophthalmic surgical procedure is cataract surgery.

10. The method of claim 1, wherein the antioxidant is administered during the ophthalmic surgical procedure.

11. The method of claim 1, wherein the antioxidant is administered before the ophthalmic surgical procedure.

12. The method of claim 1, wherein the eye oxidation is posterior capsule opacification.

13. The method of claim 1, wherein the antioxidant as administered with a pharmaceutically acceptable carrier.

14. The method of claim 1, wherein the antioxidant is administered as an eye drop.

15. The method of claim 1, wherein the antioxidant is administered from an antioxidant eye lens comprising an intraocular lens coated with an antioxidant that is implanted in the eye of the subject.
